SEQUENCE LISTING (17 sequences)

SEQ ID NO: 1 - DNA, 786 bp, Homo sapiens
atggattgggggacgctgcacactttcatcgggggtgtcaacaaacactccaccagcatcgggaaggtgtggatcacagtcatctttattttccgagtcatgatcctagtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcacaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgttgactgctttatttctaggccaacagagaagaccgtgtttaccatttttatgatttctgcgtctgtgatttgcatgctgcttaacgtggcagagttgtgctacctgctgctgaaagtgtgttttaggagatcaaagagagcacagacgcaaaaaaatcaccccaatcatgccctaaaggagagtaagcagaatgaaatgaatgagctgatttcagatagtggtcaaaatgcaatcacaggtttcccaagctaa

SEQ ID NO: 2 - DNA, 21 bp, Artificial Sequence
  Primer
atggattgggggacgctgcac

SEQ ID NO: 3 - DNA, 30 bp, Artificial Sequence
  Primer
gcagccaccactaggatcatgactcggaaa

SEQ ID NO: 4 - DNA, 20 bp, Artificial Sequence
  Primer
ctttgcccacttttgtctgt

SEQ ID NO: 5 - DNA, 22 bp, Artificial Sequence
  Primer
tgacgcagctacattttacctt

SEQ ID NO: 6 - DNA, 20 bp, Artificial Sequence
  Primer Cx30-F1
ctttgcccacttttgtctgt

SEQ ID NO: 7 - DNA, 22 bp, Artificial Sequence
  Primer Cx30-S6
tgacgcagctacattttacctt

SEQ ID NO: 8 - DNA, 20 bp, Artificial Sequence
  Primer Cx30-S2
aaccgggatgcaaaaatgtg

SEQ ID NO: 9 - DNA, 20 bp, Artificial Sequence
  Primer Cx30-S3
ttcaggcgaggagagaagag

SEQ ID NO: 10 - DNA, 20 bp, Artificial Sequence
  Primer Cx30-S6
gcttgggaaacctgtgattg

SEQ ID NO: 11 - DNA, 20 bp, Artificial Sequence
  Primer Cx30-OPL-2
gacccctctatccgaacctt

SEQ ID NO: 12 - DNA, 21 bp, Artificial Sequence
  Primer Cx30-BcII
atggattgggggacgctgcac

SEQ ID NO: 13 - DNA, 30 bp, Artificial Sequence; identical to SEQ ID NO: 3
  Primer Cx30-30mer-R

SEQ ID NO: 14 - DNA, 21 bp, Artificial Sequence
  Primer Cx30-F1BgIII
ggaagatcttttgcccacttt

SEQ ID NO: 15 - DNA, 21 bp, Artificial Sequence
  Primer Cx30-R1BgIII
ggaagatcttgacgcagctac

SEQ ID NO: 16 - DNA, 21 bp, Artificial Sequence
  Primer Cx30-F1HindIII
gcaagcttctttgcccacttt

SEQ ID NO: 17 - DNA, 21 bp, Artificial Sequence
  Primer Cx30-R1HindIII
gcaagcttgacgcagctacat

(19) United States
(12) Patent Application Publication
(10) Pub. No.: US 2004/0009505 A1
(43) Pub. Date: Jan. 15, 2004

(54) Uses of the GJB6 gene for treating certain types of alopecia including the Clouston's syndrome, and for screening compounds capable of being efficient in the treatment of alopecia genetic susceptibility

(75) Inventors: Gilles Waksman, Corbeil Essonnes (FR); Jerome Lamartine, Coucouronnes (FR)

Correspondence Address: HESLIN ROTHENBERG FARLEY & MESITI PC, 5 COLUMBIA CIRCLE, ALBANY, NY, 12203, US

(73) Assignee: Genethon III and Universite D'Evry Val D'Essonne

(21) Appl. No.: 10/400,985

(22) Filed: Mar. 27, 2003

Related U.S. Application Data
(63) Continuation of application No. PCT/FR01/02997, filed on Sep. 27, 2001.

(30) Foreign Application Priority Data
Sep. 29, 2000 (FR) FR 00.12473

(51) Int. Cl. C12Q 1/68; A01K 67/00; C07H 21/04; A61K 7/06; A61K 48/00; A61K 7/11; C12N 9/64

(52) U.S. Cl. 435/6; 514/44; 424/70.13; 435/69.1; 435/226; 435/320.1; 536/23.2; 800/8

(57) ABSTRACT

The invention is derived from the identification of mutations in the GJB6 gene, responsible for Clouston's syndrome. The symptomatology of said syndrome suggests that the GJB6 coding for connexin 30 (Cx-30), is most probably involved also in other types of alopecia with genetic susceptibility, in particular non-pathological. Therefore, the invention concerns the GJB6 gene sequence bearing at least one of the mutations 31 (G>A) and 263 (C>T), responsible for Clouston's syndrome, and the use of constructs comprising the GJB6 gene, both for preparing pharmaceutical compositions for treating Clouston's syndrome and/or certain disorders of the body hair system, and for screening molecules likely to have a beneficial effect in the treatment of alopecia. The invention also concerns methods for diagnosing Clouston's syndrome.

The present invention results from identification of mutations in the GJB6 gene, responsible for Clouston's syndrome. The symptomology of this syndrome suggests that GJB6, which encodes connexin 30 (Cx-30), is very probably also involved in other types of alopecia with genetic susceptibility, in particular nonpathological types. The invention therefore relates to the use of constructs comprising the GJB6 gene, both for preparing pharmaceutical compositions intended for the treatment of Clouston's syndrome and/or certain types of alopecia, and for screening molecules liable to have a beneficial effect in the treatment of alopecia. The invention also relates to methods for diagnosing Clouston's syndrome.

Clouston's syndrome, or Hidrotic Ectodermal Dysplasia (HED, OMIM 129500) is a rare, single-gene, autosomal dominant genetic disease. Individuals suffering from this syndrome exhibit mainly hypotrichosis and pathological alopecia, associated with dystrophy of the nails and also hyperkeratosis of the palms of the hands and of the soles of the feet. In certain cases, the patients may exhibit other clinical signs, such as deafness, mental retardation, hyperpigmentation of the skin and also abnormalities of the eye and of the skeleton.

This pathological condition affects the epidermis, which is a stratified epithelium of ectodermal origin. This epithelium consists of the basal layer, which lies on the dermo-epidermal junction, the granular and spiny layers and the uppermost horny layer. This tissue, which renews itself in two weeks, owes this property to keratinocytes, which are the main cellular constituent of the epidermis. The keratinocytes of the basal layer have the ability to self-renew, to differentiate and to begin their migration into the higher layers of the epidermis so as to reach the spiny layer and, finally, lose their nucleus in the horny layer. The capacity for renewal of keratinocytes is due to the presence of stem cells which, besides their ability to self-renew, give rise to daughter cells with a high potential for division and cells which enter into differentiation. The three populations of keratinocytes are located at the base of the epidermis. In addition, it has been shown that stem cells are also located in the hair follicle (Rochat, Kobayashi et al. 1994). These pluripotent stem cells are thought to give rise to diverse cells such as the cells of the outer sheath, the cells of the inner sheath, or of the bulb or the sebaceous gland of the hair follicle, and, finally, the interfollicular keratinocytes.

The locus responsible for Clouston's syndrome, identified in 1996, is located on chromosome 13, at 13q11 (Kibar, Der Kaloustian et al. 1996). The identification of recombination events in individuals suffering from this syndrome has made it possible to delimit the chromosomal region of interest with the centromeric microsatellite marker D13S1830 and the telomeric microsatellite marker D13S1380 (Kibar, Dube et al. 2000; Lamartine, Laoudj et al. 2000). Subsequently, a contig of BACs covering the chromosomal region of interest was obtained and a set of genes and of ESTs was located in this contig (Lamartine, Pitaval et al. 2000).

Among the genes known to be present in the HED region are the genes GJB2 and GJB6, which encode two proteins (connexin 26 and connexin 30, respectively). The connexin family comprises about fifteen members. These proteins associate with one another at the plasma membrane so as to form homomeric or heteromeric hexamers, called connexons. Two connexons form a channel connecting two cells, called gap junction. Several gap junctions form junctional plaques and are involved in the transport of small molecules such as IP₃, sugars, amino acids or else ions such as calcium. An adaptive and coordinated response of a set of cells during development or to changes in the environment is thus ensured.

Mutations in connexin genes are responsible for various hereditary pathological conditions, such as dermatological pathological conditions and pathological conditions of deafness, Charcot-Marie-Tooth neuropathy and cataracts. Several examples illustrate in particular the involvement of connexins in hereditary pathological conditions of deafness and dermatological pathological conditions.

For example, it has been demonstrated that mutations in the GJB2 gene, encoding connexin Cx-26, are responsible for a genetic form of deafness, such as DNFB1 deafness, the autosomal recessive method of transmission of which is, based on current knowledge, associated with no dermatological clinical signs. Moreover, other mutations in the gene encoding connexin Cx-26 are responsible for a form of palmoplantar keratoderma associated with deafness.

Mutations in the GJB3 gene, encoding connexin Cx-31, are also responsible for a genetic form of deafness. This is nonsyndromic neurosensory autosomal recessive deafness.

In addition, mutations of this gene are responsible for a hereditary form of dermatosis: erythrokeratoderma variabilis.

Finally, it is known that a mutation of the GJB6 gene encoding connexin Cx-30 is responsible for a form of deafness of genetic origin. This is autosomal dominant nonsyndromic sensoryneural deafness (NSRD3). A mutation of the threonine at position 5 to methionine is responsible for this pathological condition. Mutations of the same gene are, moreover, responsible for hidrotic ectodermal dysplasia, or Clouston's syndrome.

The authors of the present invention have identified the gene and the mutations causing Clouston's syndrome.

Initially, GJB2 was a very good candidate gene since it has been shown that mutations in this gene are responsible for nonsyndromic deafness of genetic origin and that deafness can be a symptom associated with Clouston's syndrome. However, no mutation in this gene has been found in the affected individuals of HED families.

More recently, another connexin (connexin 30), encoded by the GJB6 gene, was located in the 13q11 region, close to GJB2 (Kelley, Abe et al. 1999). GJB6 is a 786 base pair gene with no intron, which is expressed in particular in murine epidermis (Dahl, Manthey et al. 1996). Mutations in human connexin 30 are responsible for a form of autosomal dominant deafness (Grifa, Wagner et al. 1999). Consequently, GJB6 is also a very good candidate for Clouston's syndrome.

Sequencing of this gene, which has only one exon, made it possible to identify mutations in the coding region of the GJB6 gene, associated with Clouston's syndrome. Two types of mutation were identified in patients who are of diverse geographical origins. The mutation 31(G>A) leads to the glycine at position 11 being changed to an arginine. Another mutation, 263(C>T), causes the alanine at position 88 to be replaced with a valine. These mutations are not found in the normal individuals of the families studied nor in the general population (118 individuals were studied).

A first aspect of the invention is therefore the sequence of the human GJB6 gene, and the mutated forms of this sequence involved in Clouston's syndrome. The invention relates in particular to DNA constructs carrying the sequence of the mutated or unmutated human GJB6 gene, vectors carrying these constructs, cells transformed with these vectors, and also transgenic animals expressing wild-type or mutated human connexin 30.

The invention also relates to antisense oligonucleotides directed against the mutated alleles 31(G>A) and 263(C>T) and to gene transfer vectors intended for the treatment by gene therapy of Clouston's syndrome, and also to a method for diagnosing this syndrome, to PCR primers for detecting the mutations 31(G>A) and 263(C>T), and to kits for diagnosing Clouston's syndrome.

The inventors have therefore demonstrated the direct involvement of connexin 30 in Clouston's syndrome. One of the major clinical manifestations of Clouston's syndrome, or HED, is hypotrichosis. This hypotrichosis affects in particular the scalp, and the hair may be sparse and very fine, or even absent. It should also be noted that, when consulting the OMIM (Online Mendelian Inheritance in Man) database, the term alopecia gives access to 117 titles, including HED, which, by virtue of one of the clinical manifestations of the diseases, forms part of the types of alopecia.

It is noteworthy that connexin 30 is the only connexin identified to date as being responsible for a dermatological pathological condition for which the patients suffer from alopecia. It is therefore probable that connexin 30 is also one of the factors involved in at least some other forms of alopecia with genetic susceptibility.

It is possible to imagine two types of mutation of the GJB6 gene, responsible at least partially for types of alopecia independent of Clouston's syndrome.

A first type of mutation may affect the sequences regulating the expression of the GJB6 gene and might be the cause of alopecia, in particular in male individuals. The presence, in the regulatory regions of the GJB6 gene, of nucleotide sequences which bind factors responding to androgens might be responsible for androgenetic alopecia. Other types of sequence in the regulatory regions, which might be involved in the expression of this gene, should not, however, be excluded.

Protein factors or molecules involved in the regulation of the expression of GJB6 might act as pharmacological agents, or even as medicinal products or cosmetic products, having an effect in hair growth. Such protein factors or molecules are included in a group comprising:

molecules which inhibit or neutralize transcription factors inhibiting transcription of the GJB6 gene,

molecules which activate transcription factors activating expression of the GJB6 gene,

transcription factors involved in the repression or activation of the expression of the GJB6 gene.

The action on the GJB6 gene may be direct, i.e. on the transcription factors or the factors inhibiting them, or indirect, i.e. on the synthesis of substances or of metabolites capable of interacting with these factors.

A second type of mutation of the GJB6 gene, liable to promote a type of alopecia with genetic susceptibility concerns mutations in the coding region of the gene, which would lead to a functional loss in the Cx-30 protein. Specifically, one of the hypotheses to explain the dominant nature of Clouston's syndrome is that the mutations 31(G>A) and 263(C>T) make the product of the GJB6 gene inactive by preventing, for example, its transport to the plasma membrane (as described for mutants of connexin 32 (VanSlyke, Deschenes et al. 2000)), or its ability to form connexons. In this case, the disease might be due to a phenomenon of haploinsufficiency. It is also possible to put forward the hypothesis that these mutants are dominant negative mutants and that the presence of one or more nonfunctional subunits in a connexon is sufficient to impair or prevent its function. It is therefore possible to imagine that other mutations in the GJB6 gene may partially impair the functionality of the connexons, less drastically than the mutations involved in Clouston's syndrome, and that this partial impairment is only a factor which inhibits hair growth and/or promotes hair loss.

It should be noted that types of alopecia with genetic susceptibilities other than Clouston's syndrome are perhaps of multifactorial origin, and in particular polygenic origin. However, it is probable that certain mutations of the GJB6 gene, located in the coding sequence or in the sequences regulating the expression of this gene, are associated with types of alopecia with genetic susceptibility. The above considerations regarding the involvement of the GJB6 gene in types of alopecia in general have therefore allowed the inventors to determine several applications associated with this gene.

The present invention relates in particular to nucleic acid constructs comprising the GJB6 gene, where appropriate carrying one or more mutations observed in patients having congenital alopecia or alopecia clearly involving genetic factors. Transformed cells and transgenic animals carrying one of these constructs are also part of the invention. Such cells or transgenic animals can be used to screen molecules liable to have a beneficial effect on alopecia. Specifically, it is possible to screen molecules by bringing them into contact with cells carrying a construct such as those described above, and to then analyze the effect of said molecules on the expression of connexin 30 and/or on its functionality. The expression of connexin 30 can be determined, for example, using a DNA chip, and its functionality can be tested, for example, by measuring the electrophysiological activity of the connexons, in a system such as the Xenopus egg. Such screening methods are also part of the invention.

Moreover, the dominant nature of Clouston's syndrome suggests that the presence of one allele of the GJB6 gene carrying the mutation 31(G>A) or the mutation 263(C>T) expressed in the cells of the hair follicles is sufficient to impair the anagenic, catagenic or telogenic cycles of the hair follicle. This observation makes it possible to imagine new strategies intended to correct certain abnormalities associated with hyperpilosity (hirsutism) or, more generally, to limit the pilosity of certain regions of the body, by locally transferring a mutated allele of the GJB6 gene into the epidermal cells of the region whose pilosity it is desired to reduce. In particular, the transfer of a nucleic acid molecule encoding a form of connexin 30 carrying a nonconservative mutation at residue 11 and/or at residue 88 will lead to alopecia in the region treated. It is probable that other mutations of connexin 30 cause the same result. The invention therefore also relates to the use of a nucleic acid which allows the expression of a mutated, nonfunctional form of connexin 30, for preparing pharmaceutical and/or cosmetic compositions intended to reduce the pilosity of the treated regions.

The authors of the present invention have therefore identified and sequenced the gene responsible for Clouston's syndrome, which is the GJB6 gene, encoding connexin 30. They have shown that an individual carrying one of the mutations 31(G>A) or 263(C>T) in this gene suffers from Clouston's syndrome. The invention therefore relates, initially, to the coding sequence of the human connexin 30 gene (Seq ID No. 1), and also to a recombinant nucleic acid molecule carrying this sequence, where appropriate mutated such that the mutation leads to a nonconservative substitution of the amino acid located at position 11 and/or the amino acid located at position 88. Throughout this text, the term “sequence” denotes, firstly, the information regarding a succession of nucleotides and, secondly, the carrier of this information, i.e. a nucleic acid molecule (DNA or RNA) carrying said succession of nucleotides.

In a preferred embodiment of the nucleic acid molecules of the invention, the coding sequence of the human connexin 30 gene is placed under the control of a promoter which allows expression in eukaryotic cells, in particular those of the epidermis; preferably, this promoter is the natural promoter of the human GJB6 gene, where appropriate mutated.

Nucleic acid molecules comprising a version of the human connexin 30 gene carrying at least one mutation commonly observed in individuals having alopecia with genetic susceptibility are also part of the invention, whether the mutation(s) in question is (are) located in the coding sequence of the GJB6 gene or in the sequences regulating the expression of this gene.

The identification of mutations causing Clouston's syndrome makes it possible to envision a therapy for this pathological condition. Thus, a particular embodiment of the invention relates to an antisense oligonucleotide of a region of the coding sequence of connexin 30 comprising one of the mutations 31(G>A) or 263(C>T).

Of course, the invention also relates to gene transfer vectors comprising a nucleic acid such as those described above. The vectors may be viral vectors, for example derived from a virus selected from the group consisting of adenoviruses, retroviruses, lentiviruses, adenovirus-associated viruses (AAV), herpesviruses (HSV), the vaccinia virus, alphaviruses, baculoviruses, the SV40 virus and the Epstein-Barr virus. They may also be chimeras comprising elements of several viruses, or nonviral, optionally synthetic, vectors.

One of the aspects of the present invention is the use of a nucleic acid or of a vector such as those described above, for preparing a composition intended for the treatment of Clouston's syndrome or for the treatment of alopecia with genetic susceptibility.

Another aspect of the invention relates to pairs of primers for detecting the mutations 31(G>A) or 263(C>T), and methods for detecting these mutations.

As described in example 2 below, the mutation 31(G>A) does not affect a restriction site. On the other hand, it is possible to create one if a base close to nucleotide 31 is modified. For this, a mutagenic amplification can be carried out, for example by PCR using oligonucleotides which will modify, during the PCR, a nucleotide close to nucleotide 31. This technology makes it possible to create a restriction site which comprises said nucleotide 31. Example 2 describes a mutagenic PCR which makes it possible to create the BclI restriction site using the allele carrying the mutation 31(G>A), but which does not create a restriction site using the allele which is not mutated at position 31. The two alleles can then be identified by migration on an agarose gel after cleavage with BclI. The invention therefore relates to pairs of primers for the mutagenic amplification of a fragment of the GJB6 gene, comprising the nucleotide at position 31, such that the products of the mutagenic amplification carried out using a GJB6 allele carrying or not carrying the mutation 31(G>A) are discernible by digestion with a chosen restriction enzyme. In a particular embodiment of this aspect of the invention, the restriction enzyme is BclI, and the primers contain the following sequences:

5′-ATGGATTGGGGGACGCTGCAC-3′
and
5′-GCAGCCACCACTAGGATCATGACTCGCAAA-3′.

The invention also relates to a method of searching for the mutation 31(G>A), for diagnosing Clouston's syndrome, comprising the following steps:

amplification of a fragment of the GJB6 gene with a pair of primers such as those described above,

digestion of the amplification product with the appropriate restriction enzyme, which is BclI in the particular case in which the amplification has led to the creation of a BclI restriction site using the allele carrying the mutation 31(G>A),

analyzing the digestion product.

The mutation 263(C>T) eliminates an HaeII restriction site which exists on the wild-type allele. The mutation can therefore be revealed by amplification of the region containing the mutation, followed by cleavage of the amplification product with the HaeII restriction enzyme. The two alleles can then be separated by migration on an agarose gel. The invention therefore relates to pairs of primers for amplifying a fragment of the GJB6 gene, comprising the nucleotide at position 263, chosen such that digestion of the amplified fragment with HaeII makes it possible to distinguish the presence or absence of the HaeII restriction site between nucleotides 260 and 270. In particular, the invention relates to a pair of primers of 20 to 50 nucleotides comprising the following sequences:

5′-CTTTGCCCACTTTTGTCTGT-3′
and
5′-TGACGCAGCTACATTTTACCTT-3′.

These pairs of primers can be used in a method of searching for the mutation 263(C>T), for diagnosing Clouston's syndrome, comprising the following steps:

amplification of a fragment of the GJB6 gene with a pair of primers such as those described above,

digestion of the amplification product with the HaeII enzyme,

analysis of the digestion product, the presence of the mutated allele leading to a band which is not cleaved by HaeII.

The pairs of primers described above can be included in a kit for diagnosing Clouston's syndrome, which is also part of the invention. Such a kit may contain one or more pairs of primers. It may also comprise the BclI and HaeII restriction enzymes and buffers suitable for the activity of these enzymes.

Knowledge of mutations involved in a dominant single-gene pathological condition, one of the symptoms of which is alopecia, may have other applications. Thus, a nucleic acid or a vector such as those described above and carrying a mutated version of the GJB6 gene, leading, for example, to a nonconservative mutation of residue 11 or of residue 88, can also be used, according to the present invention, to prepare a composition intended to limit hair growth and/or to promote hair loss. Other mutations leading to a loss of functionality of connexin 30 can also be envisioned in this aspect of the invention.

The present invention therefore also comprises any pharmaceutical and/or cosmetic composition comprising a nucleic acid molecule and/or a vector such as those described above. Depending on the case, this composition is intended for the treatment of Clouston's syndrome, for the treatment of other types of alopecia with genetic susceptibility, or for the reduction of the pilosity of certain regions of the body.

The present invention also relates to the recombinant proteins encoded by the nucleic acids and the vectors described above, and also to any pharmaceutical and/or cosmetic composition comprising one or more of these recombinant proteins. Where appropriate, these proteins may be truncated. A composition of the invention may also contain only fragments of the recombinant proteins described above.

Preferably, the compositions of the invention can be administered by external or local application. They may, for example, be a cream, a gel or a spray.

Another aspect of the invention relates to eukaryotic cells comprising a recombinant DNA construct such as those described above. In these cells, the recombinant DNA construct which allows expression of the human connexin 30 gene may be present in extrachromosomal form or may be integrated into the genome of said cells. In a particular embodiment of this aspect of the invention, the cells comprising a recombinant DNA construct which allows expression of the human connexin gene are Xenopus egg or HeLa cells. In the cells of the invention, the expression of human connexin 30 may be stable or transient, constitutive or inducible.

The invention also relates to a transgenic animal comprising cells expressing the human connexin 30 gene, in its wild-type form or in a mutated form, such as the cells described above. The expression of the human connexin 30 gene in an animal of the invention may or may not be tissue-specific, and may be constitutive, inducible or repressible.

The cells and transgenic animals of the invention may in particular be used to screen molecules which are effective in the treatment of certain types of alopecia. Also part of the invention is therefore a method for screening molecules which are effective in the treatment of certain types of alopecia, comprising the following steps:

bringing the candidate molecule into contact with a cell or with an animal according to the invention,

determining the effect of the candidate molecule on the level of expression of the GJB6 gene and/or on the activity of the connexin 30.

In this method, the step for determining the effect of the candidate molecule on the level of expression of the GJB6 gene may be carried out using a DNA chip.

In a particular embodiment of the method of the present invention, the cell carrying an allele of the human connexin 30 gene is a Xenopus egg, and the step for determining the effect of the candidate molecule on the activity of the connexin 30 is carried out by measuring the electrophysiological activity of the connexons.

The examples and figures given below, in a nonlimiting capacity, illustrate and specify certain aspects of the present invention.

Legends of the figures:

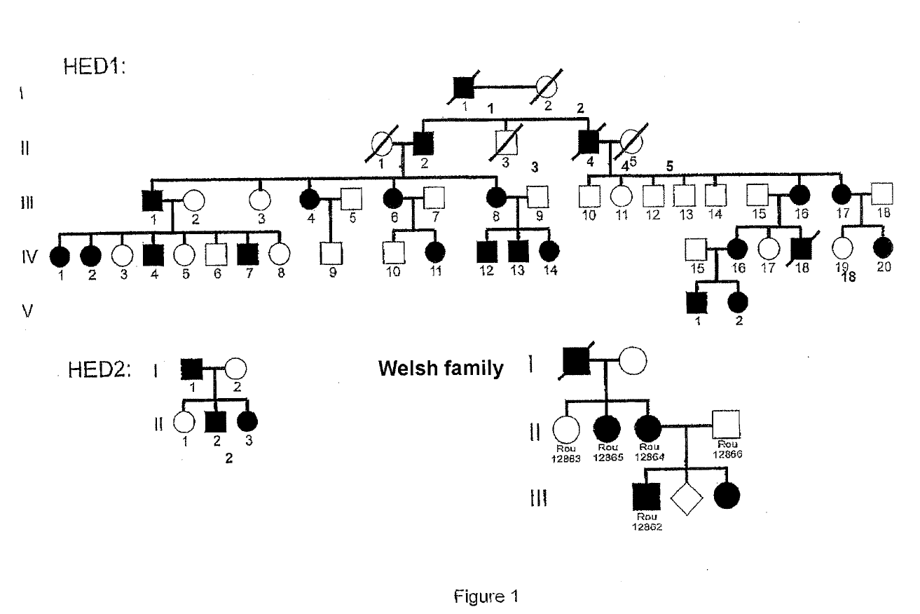

FIG. 1: Genealogical tree of the French families (HED1 and HED2) and of the Welsh family analyzed in this study. The numbers in gray represent the individuals whose DNA was analyzed.

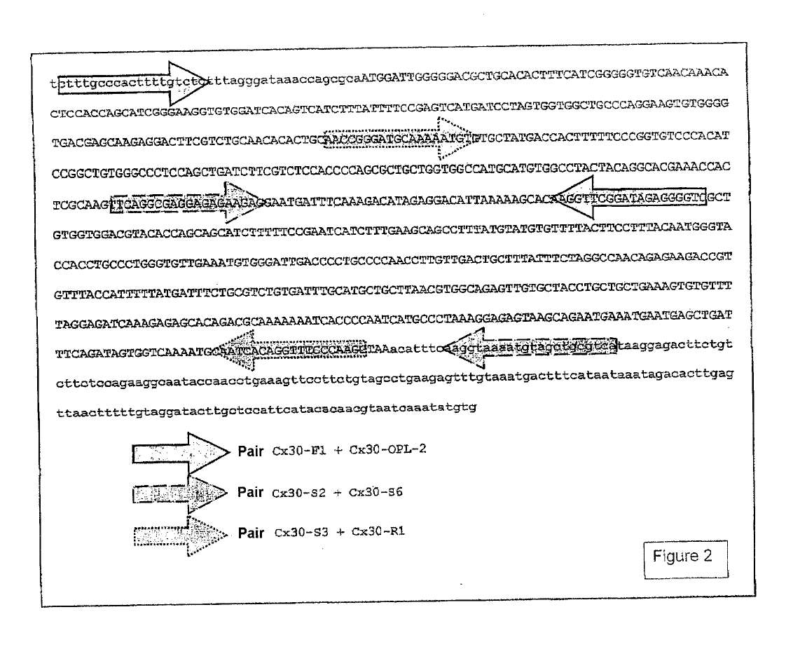

FIG. 2: Sequence of the GJB6 gene with the position of the various primers used. The coding region is represented in upper case.

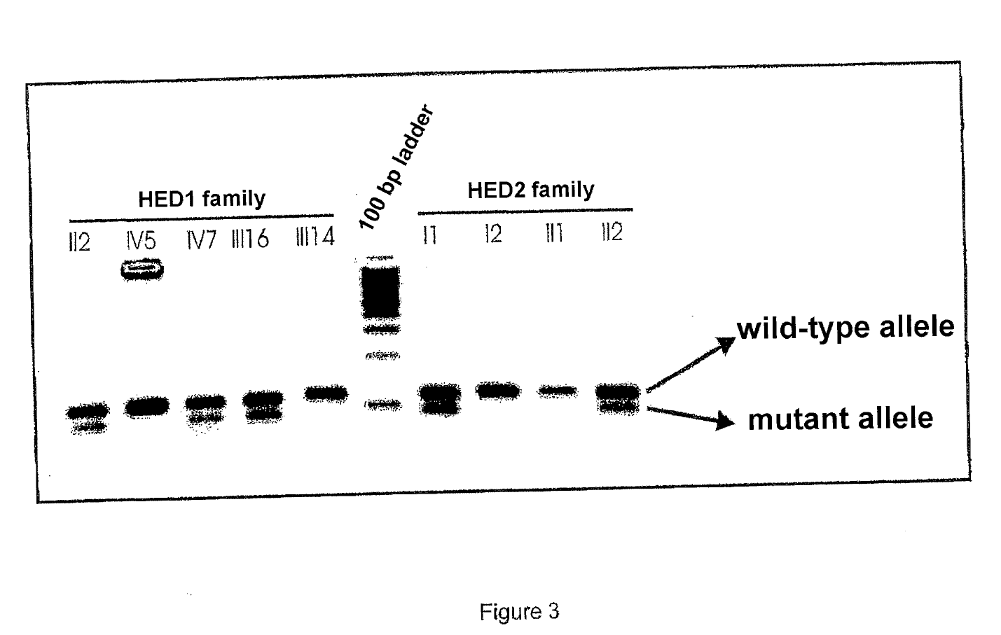

FIG. 3: Genotyping of sick and healthy individuals from two families carrying the mutation 31(G>A). The product of amplification by mutagenic PCR of the mutant allele is cleaved with BclI and gives two fragments (80 bp and 39 bp, which is not visible on the photograph). The product of amplification by mutagenic PCR of the wild-type allele is not cleaved (119 bp).

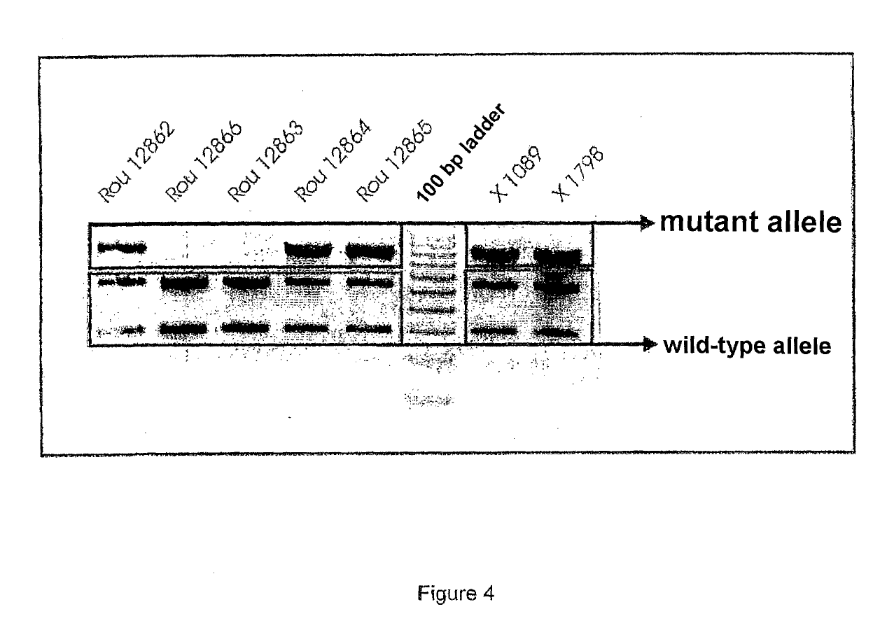

FIG. 4: Genotyping of individuals from families carrying the mutation 263(C>T). The product of PCR amplification of the mutant allele (not cleaved) is 854 bp in length. The product of PCR amplification of the wild-type allele is cleaved with HaeII and gives two fragments (304 bp and 550 bp).

### EXAMPLE 1

### The search for Mutations in GJB6

Genetic Material Used:

DNA samples from healthy and affected individuals, of diverse geographical origins, were used (see Table 1). This collection comprises in particular samples from two unrelated French families (HED1 and HED2) carrying the disease (see FIG. 1). The unrelated individuals who were used as a negative control come from several independent families of the CEPH (Centre d'Etude du Polymorphisme Humain) [Centre for the Study of Human Polymorphism].

TABLE 1
Samples from healthy (H) or affected (A) individuals
obtained through several collaborations

| Sample | Status | Origin |
|---|---|---|
| X1111 | A | India |
| X1234 | A | Scotland/Ireland |
| X1239 | H | Scotland/Ireland |
| X1794 | A | South Africa |
| Rou6206 | H | Canada (Quebec) 1 |
| Rou6209 | A | Canada (Quebec) 1 |
| Rou7571 | H | Canada (Quebec) 2 |
| Rou7572 | A | Canada (Quebec) 2 |
| Rou8946 | A | Spain |
| Rou8950 | H | Spain |
| Rou9387 | H | France |
| Rou9455 | A | France |
| Rou11442 | A | Canada (Quebec) 3 |
| Rou11444 | H | Canada (Quebec) 3 |
| Rou12862 | A | Wales |
| Rou12866 | H | Wales |
| Rou12863 | H | Wales |
| Rou12864 | A | Wales |
| Rou12865 | A | Wales |
| X1089 | A | Wales |
| X1798 | A | Malaysia |

Sequencing of the GJB6 Gene:

The initial search for mutations in the GJB6 gene was carried out by sequencing three PCR fragments which cover the entire gene with no intron (pairs F1/OPL2, S2/S6 and S3 μl; see FIG. 2 and Table 2). The DNAs of a healthy individual and of a sick individual from each of the two families studied, and also those of the individuals Rou12866 and Rou12862 (see Table 1), were sequenced. This work was carried out in collaboration with the sequencing department of Genethon.

TABLE 2

Sequences of the various primers used
to sequence the GJB6 gene

| | |
|---|---|
| Primer | Sequence |

| | |
|---|---|
| Cx30-F1 | 5′-CTTTGCCCACTTTTGTCTGT-3′ |
| Cx30-R1 | 5′-TGACGCAGCTACATTTTACCTT-3′ |
| Cx30-S2 | 5′-AACCGGGATGCAAAAATGTG-3′ |
| Cx30-S3 | 5′-TTCAGGCGAGGAGAGAAGAG-3′ |
| Cx30-S6 | 5′-GCTTGGGAAACCTGTGATTG-3′ |
| Cx30-OPL-2 | 5′-GACCCCTCTATCCGAACCTT-3′ |

Results:

Analysis of the sequences of the DNAs from the individuals C.1 (sick) and III.15 (healthy) of the HED1 family (see FIG. 1) made it possible to show that the first is heterozygous for a point mutation 31(G>A) which affects the first base of codon 11 of the sequence and leads to substitution of a glycine residue with an arginine residue.

Moreover, sequencing of the entire coding sequence of the GJB6 gene from an affected individual (Rou12862) and from a healthy individual (Rou12866) of a family not exhibiting the mutation 31(G>A) made it possible to identify, in this family, a new mutation, 263(C>T), in this same gene. This mutation affects the second base codon 89 of the sequence and leads to substitution of an alanine residue with a valine residue.

### EXAMPLE 2

### Confirmation of the Mutations in GJB6

Principle of Genotyping a Large Number of Individuals for the Mutations:

The genotyping of a large number of individuals should be carried out using a simple technique for detecting already known mutations. The method used here consists in demonstrating the creation or disappearance of a restriction site in the mutated sequence. Two techniques were used:

Mutation 263(C>T): the mutation eliminates an HaeII restriction site existing on the wild-type allele. Demonstration of the mutation is performed by PCR amplification of the region containing the mutation and then by cleavage of the amplification product with the appropriate restriction enzyme. The two alleles can then be separated by migration on an agarose gel.

Mutation 31(G>A): the mutation does not affect a restriction site. On the other hand, it is possible to create one if a base close to the mutated site is also modified. Mutagenic oligonucleotides were used here, to modify, during the PCR, a nucleotide close to the mutation, so as to create the BclI restriction site. The two alleles can then be identified by migration on an agarose gel after cleavage with the appropriate enzyme.

Mutation 31 (G>A):

Once the mutation 31(G>A) had been identified, the mutations in the other individuals were sought using a mutagenic PCR (Cx30-BclI/Cx30-30mer-R; see Table 3), which creates a BclI restriction site in the mutated allele. The PCR product is then digested with the BclI enzyme. Migration on the gel (2% agarose+2% Nusieve®) makes it possible to distinguish between the mutated allele and the wild-type allele by virtue of a difference in size of 39 base pairs.

TABLE 3

Sequence of the various primers used
to amplify the GJB6 gene or fragments of this gene

| | |
|---|---|
| Primer | Sequence |

| | |
|---|---|
| Cx30-BclI | 5′-ATGGATTGGGGGACGCTGCAC-3′ |
| Cx30-30mer-R | 5′-GCAGCCACCACTAGGATCATGACTCGGAAA-3′ |
| Cx30-F1BglII | 5′-GGAAGATCTTTTGCCCACTTT-3′ |
| Cx30-R1BglII | 5′-GGAAGATCTTGACGCAGCTAC-3′ |
| Cx30-F1HindIII | 5′-GCAAGCTTCTTTGCCCACTTT-3′ |
| Cx30-R1HindIII | 5′-GCAAGCTTGACGCAGCTACAT-3′ |

All the available samples of the two French HED families were tested using this mutagenic PCR. All the affected individuals proved to be heterozygous, while the healthy individuals are homozygous (see FIG. 3).

This same method made it possible to detect the mutation 31(G>A) in other affected individuals of diverse geographical origins (see Table 4).

Mutation 263(C>T):

After its discovery in the individual Rou12862, the mutation 263(C>T) was sought in the other individuals according to the technique described above. The mutation eliminates an HaeII restriction site existing on the wild-type allele. Distinction between the two alleles is made after PCR (Cx30-F1/Cx30-R1; see Table 3) and enzyme cleavage with HaeII. The heterozygous individuals exhibit a band corresponding to the undigested PCR product in addition to the smaller bands corresponding to the wild-type allele.

Simple cleavage of the products of PCR Cx30-F1/Cx30-R1 with the HaeII enzyme (see FIG. 4) therefore made it possible to verify that all of the affected individuals who did not carry the mutation 31(G>A) exhibited the mutation 263(C>T) and that the latter is absent in the healthy individuals (see Table 4).

Genotyping of the Controls

In order to verify that these two mutations do not correspond to polymorphism, the DNA of a large number of independent healthy individuals were studied using the two strategies described above. The 93 individuals tested are the grandparents of 24 families of the CEPH. The individuals tested exhibit neither of the two mutations. Since the mutation 263(C>T) was identified in a family of Indian origin, 21 healthy independent Indians were tested. These individuals exhibit neither of the two mutations.

TABLE 4
Genotyping of the DNAs obtained through collaboration

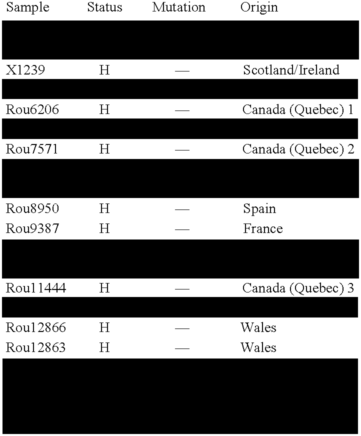

A = affected, H = healthy

### EXAMPLE 3

### Cloning of the Wild-Type and Mutant Alleles of Connexin 30

With the aim of carrying out functional studies on the wild-type and mutated connexins 30 in various eukaryotic cell systems, the various alleles of the GJB6 gene was cloned into the vector pUC19, flanked by the BglII or HindIII restriction sites.

In a first step, the gene was amplified by PCR using primers containing BglII or HindIII restriction sites close to the 5′ ends (Cx30-F1 BglII/Cx30-R1 BglII or Cx30-F1 HindIII/Cx30-R1 HindIII, see Table 3). Once the gene had been amplified (from genomic DNA originating from sick, heterozygous individuals carrying each of the two mutations), the ends of the amplification product were made blunt by the action of the DNA polymerase I Klenow fragment. The PCR product was cloned into the vector pUC19 (linearized with an enzyme giving blunt ends). After ligation and transformation in the E. coli strain JM109, the recombinants were identified by PCR using the primers M13-21 and M13-40 of the vector. The nature of the insert (mutated or wild-type) was determined using the techniques described in Example 2.

Six different clones were obtained, corresponding to the wild-type form and to the mutated forms 31(G>A) and 263(C>T), bordered by BglII or HindIII sites.

### REFERENCES

E. Dahl, D. Manthey, et al. (1996). “Molecular cloning and functional expression of mouse connexin-30, a gap junction gene highly expressed in adult brain and skin [published erratum appears in J Biol Chem Oct. 18, 1996; 271(42):26444].” J Biol Chem 271(30): 17903-10.

A. Grifa, C.A. Wagner, et al. (1999). “Mutations in GJB6 cause nonsyndromic autosomal dominant deafness at DFNA3 locus [letter].” Nat Genet 23(1): 16-8.

P. M. Kelley, S. Abe, et al. (1999). “Human connexin 30 (GJB6), a candidate gene for nonsyndromic hearing loss: molecular cloning, tissue-specific expression, and assignment to chromosome 13q12.” Genomics 62(2): 172-6.

Z. Kibar, V. M. Der Kaloustian, et al. (1996). “The gene responsible for Clouston hidrotic ectodermal dysplasia maps to the pericentromeric region of chromosome 13q.” Hum Mol Genet 5(4): 543-7.

Z. Kibar, M. P. Dube, et al. (2000). “Clouston hidrotic ectodermal dysplasia (HED): genetic homogeneity, presence of a founder effect in the French Canadian population and fine genetic mapping [In Process Citation].” Eur J Hum Genet 8(5): 372-80.

J. Lamartine, D. Laoudj, et al. (2000). “Refined localization of the gene for Clouston syndrome (hidrotic extodermal dysplasia) in a large French family.” Br J Dermatol 142(2): 248-52.

J. Lamartine, A. Pitaval, et al. (2000). “A 1.5-Mb physical map of the hidrotic ectodermal dysplasia (Clouston syndrome) gene region on human chromosome 13q11.” Genomics 67(2): 232-6.

A. Rochat, K. Kobayashi, et al. (1994). “Location of stem cells of human hair follicles by clonal analysis.” Cell 76(6): 1063-73.

J. K. VanSlyke, S. M. Deschenes, et al. (2000). “Intracellular transport, assembly, and degradation of wild-type and disease-linked mutant gap junction proteins.” Mol Biol Cell 11(6): 1933-46.

1. (Original) A recombinant nucleic acid carrying the coding sequence of the human connexin 30 gene (Seq ID No. 1), characterized in that SEQ ID No. 1 comprises a mutation 31(G>A) and/or a mutation 263(C>T) leading, respectively, to a nonconservative substitution of the amino acid located at position 11 and/or of the amino acid located at position 88.

2. (Original) The nucleic acid as claimed in claim 1, characterized in that the coding sequence of the human connexin 30 gene is placed under the control of a promoter which allows its expression in eukaryotic cells.

3. (Original) The nucleic acid as claimed in claim 2, characterized in that the promoter controlling the expression of the human connexin 30 gene is the natural promoter of the human GJB6 gene, where appropriate mutated.

4. (Original) An antisense oligonucleotide of a region of the sequence of the nucleic acid of claim 1, comprising one of the mutations 31(G>A) or 263(C>T).

5. (Previously amended) A gene transfer vector comprising the nucleic acid of claim 1.

6. (Original) The vector as claimed in claim 5, characterized in that it is a recombinant virus.

7. (Original) The vector as claimed in claim 5, characterized in that it is a nonviral vector for gene transfer.

8. (Cancelled)

9. (Cancelled)

10. (Previously amended) A eukaryotic cell comprising a recombinant DNA construct as claimed claim 1.

11. (Original) The cell as claimed in claim 10, such that the recombinant DNA construct which allows the expression of the human connexin 30 gene is present in extrachromosomal form.

12. (Original) The cell as claimed in claim 10, such that the recombinant DNA construct which allows the expression of the human connexin 30 gene is integrated into the genome of said cell.

13. (Previously amended) The cell as claimed in claim 10, characterized in that it is a Xenopus egg or HeLa cell.

14. (Previously amended) A transgenic animal comprising cells according to claim 10.

15. (Previously amended) A method for screening molecules which are effective in the treatment of certain types of alopecia, comprising the following steps:
bringing the candidate molecule into contact with a cell as claimed in claim 10,
determining the effect of the candidate molecule on the level of expression of the GJB6 gene and/or on the activity of the connexin 30.

16. (Original) The method as claimed in claim 15, in which the step for determining the effect of the candidate molecule on the level of expression of the GJB6 gene is carried out using a DNA chip.

17. (Original) The method as claimed in claim 15, in which the cell carrying an allele of the human connexin 30 gene is a Xenopus egg, and the step for determining the effect of the candidate molecule on the activity of the connexin 30 is carried out by measuring the electrophysiological activity of the connexons.

18. (Currently amended) A pair of primers for the mutagenic amplification of a fragment of the GJB6 gene, comprising the nucleotide at position 31, such that the products of the mutagenic amplification carried out using a GJB6 allele carrying or not carrying the mutation 31 (G>A) are discernible by digestion with a chosen restriction enzyme, characterized in that the restriction enzyme is BclI, and in that the primers contain the following sequences:

| | |
|---|---|
| 5′-ATGGATTGGGGGACGCTGCAC-3′ | (SEQ ID NO. 2) |
| and | |
| 5′-GCAGCCACCACTAGGATCATGACTCGGAAA-3′. | (SEQ ID NO. 3) |

19. (Currently amended) A pair of primers for amplifying a fragment of the GJB6 gene, comprising the nucleotide at position 263, characterized by the following sequences:

| | |
|---|---|
| 5′-CTTTGCCCACTTTTGTCTGT-3′ | (SEQ ID NO. 4) |
| and | |
| 5′-TGACGCAGCTACATTTTACCTT-3′ | (SEQ ID NO. 5). |

20. (Original) A method of searching for the mutation 31 (G>A), in which
the amplification of a fragment of the GJB6 gene is carried out with a pair of primers as claimed in claim 18,
the amplification product is digested with the BclI enzyme,
the presence of the mutated allele leads to a difference of 39 base pairs.

21. (Original) A method of searching for the mutation 263(C>T), for diagnosing Clouston's syndrome, comprising the following steps:
amplification of a fragment of the GJB6 gene with a pair of primers as claimed in claim 19,
digestion of the amplification product with the HaeII enzyme,
analysis of the digestion product, the presence of the mutated allele leading to a band which is not cleaved by HaeII.

22. (Previously amended) A kit for diagnosing Clouston's syndrome, comprising one or more pairs of primers as claimed in claim 18.

23. (Original) The diagnostic kit as claimed in claim 22, also comprising the BclI and HaeII restriction enzymes and buffers suitable for the activity of these enzymes.

24. (Previously amended) A recombinant protein encoded by a nucleic acid molecule as claimed in claim 1.

25. (Previously amended) A pharmaceutical and/or cosmetic composition for the prevention and/or treatment of disorders of the hair system, comprising a recombinant protein as claimed in claim 24.

26. (Original) The use of a recombinant nucleic acid carrying the coding sequence of the human connexin 30 gene (Seq ID 1), for producing a composition intended for the treatment of Clouston's syndrome or alopecia with genetic susceptibility.
